(19) United States
(12) Patent Application Publication
(10) Pub. No.: US 2026/0191894 A1
(43) Pub. Date: Jul. 9, 2026

(54) TREATMENTS FOR EYE DISORDERS

(71) Applicant: PS Therapy, Inc., Westmont, IL (US)

(72) Inventor: Siddharth Narendran, Tamil Nadu (IN)

(21) Appl. No.: 19/271,133

(22) Filed: Jul. 16, 2025

(30) Foreign Application Priority Data
Jul. 16, 2024 (IN) 202441054157

(51) Int. Cl. A61K 31/7048; A61K 31/047; A61K 31/192; A61K 31/352; A61K 31/404; A61P 27/02

(52) U.S. Cl. CPC A61K 31/7048; A61K 31/047; A61K 31/192; A61K 31/352; A61K 31/404; A61P 27/02

(57) ABSTRACT

The present invention is directed to compositions for oral administration comprising two or more active ingredients selected from the group consisting of quercetin, metabolites, derivatives and salts and hydrates thereof, a kaempferol rutinoside and salts and hydrates thereof, indole-3-carbinol and derivatives and metabolites and salts and hydrates thereof and gallic acid and salts and hydrates thereof. The present invention is further directed to methods for treating dry eye disease, treating age-related macular degeneration, increasing Bruch's membrane stiffness, modulating expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof, increasing adhesion of retinal pigment epithelium cells to a Bruch's membrane, and methods of modulating a secretory profile of senescent cells exhibiting the senescence-associated secretory phenotype comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

### FIELD OF THE INVENTION

The present invention is directed to compositions for oral administration comprising two or more active ingredients selected from the group consisting of quercetin, metabolites, derivatives and salts and hydrates thereof, a kaempferol rutinoside and salts and hydrates thereof, indole-3-carbinol and derivatives and metabolites and salts and hydrates thereof and gallic acid and salts and hydrates thereof.

The present invention is further directed to methods for treating dry eye disease, treating age-related macular degeneration, increasing Bruch's membrane stiffness, modulating expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof, increasing adhesion of retinal pigment epithelium cells to a Bruch's membrane and methods of modulating a secretory profile of senescent cells exhibiting the senescence-associated secretory phenotype comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

### BACKGROUND OF THE INVENTION

Eye conditions including dry eye disease afflict 100's of millions of people worldwide. Dry eye disease is an ocular disease affecting approximately 10-20% of the population. Over 350 million people worldwide suffer from visual impairment or blindness due to retinal diseases and disorders. These numbers are only expected to rise due to aging populations in many countries. Despite the huge population of afflicted individuals there are a dearth of treatments or preventative medicines available to combat dry eye disease.

As mentioned above, dry eye disease likely afflicts over a billion people worldwide. In addition, various subsets of this population, including contact lens wearers and patients undergoing glaucoma treatment for example, have a high incidence of dry eye. In individuals suffering from dry eye, the protective layer of tears that normally protects the ocular surface is compromised. This compromise is a result of insufficient or unhealthy production of one or more tear components. These insufficiencies are typically categorized as reduced aqueous synthesis, known as aqueous deficiency, or lipid layer deficient, known as meibomian gland deficiency (“MGD”). These deficiencies can lead to compromised tear layer exposure of the surface of the eye, ultimately promoting desiccation and damage of surface cells.

Signs and symptoms of dry eye disease include but are not limited to conjunctival and corneal staining, redness, blurry vision, decreased tear film break-up time, decreased tear production, tear volume, and tear flow, abnormal tear layer composition, increased conjunctival redness, excess debris in the tear film, ocular dryness, ocular grittiness, ocular burning, foreign body sensation in the eye, excess tearing, photophobia, ocular stinging, refractive impairment, ocular sensitivity, and ocular irritation. Patients may experience one or more of these symptoms. The excess tearing response may seem counterintuitive to dry eye, however excess tearing is a natural response to the irritation and foreign body sensation caused by dry eye. Some people may also experience ocular itching due to a combination of ocular allergies and dry eye.

There are many possible variables that can influence a person's signs or symptoms of dry eye including levels of circulating hormones, various autoimmune diseases (e.g. Sjögren's syndrome and systemic lupus erythematosus), cutting of sensory corneal nerve plexus reducing corneal sensation such as from ocular surgeries including PRK or LASIK®, many medications, environmental conditions, visual tasking with reduced blinking such as computer use, ocular fatigue, and mechanical influences such as reduced corneal sensitivity, partial lid closure during sleep, surface irregularities (e.g. pterygium), and other lid or ocular surface irregularities (e.g. ptosis, entropion/ectropion, pinguecula).

There are a number of products commercially available for the treatment of dry eye. However, such products provide only temporary relief of acute symptoms. These products typically last for a few minutes and in rare cases up to ten minutes. Further, these product provide virtually none of the known inotropic excipients present in tears that promote the healthiest ocular surface, such as growth factors, lipid solubilizers, antimicrobials and other anti-inflammatory components of a healthy normal tear film. Artificial tears are suitable for short term use only and provide virtually no long-term health benefit. In addition, these products may themselves cause ocular discomfort upon installation in the eye and or with prolonged or chronic repeat use. For example, artificial tears and ointments may provide temporary relief of dry eye but do little to arrest or reverse any damaging conditions.

For more severe cases of dry eye, in which the cornea is inflamed, anti-inflammatory agents are sometimes prescribed. Topical corticosteroids (in eye drops) are safe for short-term use to combat inflammation, but can cause side effects, including but not limited to decreased wound healing, cataract, and in some cases, increased risk of elevated intra-ocular pressure in patients. Likewise, nonsteroidal anti-inflammatory drugs (NSAIDs) in their current ophthalmic dosage forms are approved for short term use only. For example, the use of NSAIDs to treat inflammation and pain associated with post ocular-surgery may result in corneal damage in patients predisposed to such conditions. Further the use of NSAIDs may delay wound healing after repeated dosing, or ocular discomfort.

Commercial cyclosporin-A (Restasis®-Allergan) is the first approved therapeutic agent for the treatment of dry eye and is suitable for long term use. However, the primary side effect cited on the package insert is ocular burning and stinging upon instillation, and Restasis® was shown to be effective in only 17% of patients. To improve patient discomfort during the induction phase of cyclosporin therapy, clinicians may prescribe topical corticosteroids or NSAIDs (in eye drop form) in conjunction with cyclosporin-A. However, it takes about six months for just 15% of dry eye patients treated to demonstrate a 10 mm improvement in their tearing as measured by experts in the art versus vehicle. Autologous serum, on the other hand, in which blood is drawn from a patient and cells are spun off harvesting the plasma which is then frozen until use, contains within most if not all of the beneficial inotropic factors found in tears. Autologous serum has been the most effective treatment to date for severe dry eye such as that associated with Sjogren's syndrome.

Macular degeneration is a disease of the eye that results in minor to severe impairment of the subject's sharp central vision, which is necessary for activities such as reading and driving. Age-related macular degeneration (“AMD”) afflicts an estimated 30 to 50 million people worldwide and is the leading cause of severe vision loss in Western societies. AMD disrupts the photoreceptors of the macula in one of two ways: (1) deposits of extracellular debris between Bruch's membrane and the retinal pigment epithelium known as “dry” macular degeneration and (2) breaks in Bruch's membrane that allow angiogenic blood vessels from the choroid to penetrate the retinal pigment epithelium known as “wet” macular degeneration. Dry AMD progresses slowly and is responsible for about 90% of AMD worldwide. Wet AMD can be sudden, severe and irreversible due to bleeding and scarring of the macular region including the fovea. Although wet AMD accounts for only 10% of AMD worldwide it is responsible for 90% of AMD-associated blindness.

Treatments for wet AMD include anti-angiogenic drugs. These anti-angiogenic drugs include vascular endothelial growth factor (“VEGF”) inhibitors including aflibercept, bevacizumab, ranibizumab and faricimab. Additional treatment methods include photodynamic therapy and laser therapy. However, despite available treatments there continue to be patients who become legally blind due to AMD.

Kaempferol and quercetin and many of their derivatives are naturally occurring compounds known as a flavonol. Kaempferol and quercetin have been shown to reduce the risk of cancer. However, kaempferol and quercetin and many of its derivatives have not been studied to treat dry eye and glaucoma.

Indole-3-carbinol is a metabolite of glucosinolates found in cruciferous vegetables. Gallic acid is a naturally occurring phenolic acid found in many plants. Neither indole-3-carbinol or gallic acid has been studied to treat dry eye and glaucoma.

Thus, there is a need in the art for additional therapeutic treatments for eye disorders such as dry eye and AMD.

### SUMMARY OF THE INVENTION

The present invention is directed to a composition for oral administration comprising two or more active ingredients selected from the group consisting of quercetin, metabolites, derivatives and salts and hydrates thereof, a kaempferol rutinoside and salts and hydrates thereof, indole-3-carbinol and derivatives and metabolites and salts and hydrates thereof and gallic acid and salts and hydrates thereof.

The present invention is further directed to methods for treating dry eye disease comprising administering a composition of the present invention to a subject in need thereof.

The present invention is further directed to methods for treating dry eye disease comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

The present invention is further directed to methods for treating age-related macular degeneration comprising administering a composition of the present invention to a subject in need thereof.

The present invention is further directed to methods for treating age-related macular degeneration comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

The present invention is further directed to methods for increasing Bruch's membrane stiffness comprising administering a composition of the present invention to a subject in need thereof.

The present invention is further directed to methods for increasing Bruch's membrane stiffness comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

The present invention is further directed to methods for modulating expression of a gene selected from the group consisting of YAP, TAZ, integrin α5, integrin β5, interferon β1, interleukin-6, C-X-C motif chemokine ligand 1, C-X-C motif chemokine ligand 10 and combinations thereof comprising administering a composition of the present invention to a subject in need thereof.

The present invention is further directed to methods for modulating expression of a gene selected from the group consisting of YAP, TAZ, integrin α5, integrin β5, interferon β1, interleukin-6, C-X-C motif chemokine ligand 1, C-X-C motif chemokine ligand 10 and combinations thereof comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

The present invention is further directed to methods for increasing adhesion of retinal pigment epithelium cells to a Bruch's membrane comprising administering a composition of the present invention to a subject in need thereof.

The present invention is further directed to methods for increasing adhesion of retinal pigment epithelium cells to a Bruch's membrane comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

The present invention is further directed to methods of modulating a secretory profile of senescent cells in a subject comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof, wherein the senescent cells of the subject display the senescence-associated secretory phenotype prior to administration of the one or more active ingredients.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C and 1D. Slit lamp analysis of fluorescein-stained corneal epithelium showing untreated (1A) and (1C) mouse eyes and mouse eyes treated with 100 micromolar of indole-3-carbinol (1B) and 1 millimolar of indole-3-carbinol (1D), wherein the mouse eyes were previously treated with scopolamine.

FIG. 2. Bar chart demonstrating the fold change from control of ICAM-1 mRNA expression levels wherein I3C denotes indole-3-carbinol, LE denotes the untreated left mouse eye and RE denotes the treated right mouse eye.

FIG. 3. Bar chart demonstrating the stiffness in kiloPascals of the macular region of the Bruch's membrane in normal human cadaver eyes, human cadaver eyes under age-related macular degeneration (“AMD”) and human cadaver eyes under geographic atrophy (“GA”).

FIG. 4. Bar chart demonstrating the percent area of the macular region of the Bruch's membrane (“MBrM”) covered by retinal pigment epithelial (“RPE”) cells in normal human cadaver eyes and eyes under age-related macular degeneration (“AMD”).

FIGS. 5A and 5B. 5000× Scanning Electron Microscope images showing the Bruch's membrane seeded with untreated (5A)- and kaempferol 3-O-β-rutinoside treated (5B)-primary cultured retinal pigment epithelial cells.

FIG. 6. Bar chart demonstrating the percentage resurfacing of Buch's membrane by retinal pigment epithelial cells with or without treatment with kaempferol 3-O-β-rutinoside.

FIGS. 7A, 7B and 7C. 5000× Scanning Electron Microscope images showing the normal Bruch's membrane seeded with untreated (7A), 1× mixture of kaempferol 3-O-β-rutinoside, quercetin-3-glucoside and gallic acid treated (7B) and 0.01× mixture of kaempferol 3-O-β-rutinoside, quercetin-3-glucoside and gallic acid treated (7C) primary cultured retinal pigment epithelial cells.

FIG. 8. Bar chart demonstrating the percentage resurfacing of normal Buch's membrane by retinal pigment epithelial cells with or without treatment with a mixture of kaempferol 3-O-β-rutinoside, quercetin-3-glucoside and gallic acid.

FIGS. 9A, 9B and 9C. 5000× Scanning Electron Microscope images showing the AMD afflicted Bruch's membrane seeded with untreated (9A), 1× mixture of kaempferol 3-O-β-rutinoside, quercetin-3-glucoside and gallic acid treated (9B) and 0.01× mixture of kaempferol 3-O-β-rutinoside, quercetin-3-glucoside and gallic acid treated (9C) primary cultured retinal pigment epithelial cells.

FIG. 10. Bar chart demonstrating the percentage resurfacing of AMD afflicted Buch's membrane by retinal pigment epithelial cells with or without treatment with a mixture of kaempferol 3-O-β-rutinoside, quercetin-3-glucoside and gallic acid.

FIGS. 11A and 11B. 5000× Scanning Electron Microscope images showing the normal Bruch's membrane seeded with untreated (11A)- and 0.01× indole-3-carbinol treated (11B) primary cultured retinal pigment epithelial cells.

FIGS. 12A, 12B and 12C. 5000× Scanning Electron Microscope images showing the AMD afflicted Bruch's membrane seeded with untreated (12A), 1× indole-3-carbinol treated (12B) and 0.01× indole-3-carbinol treated (12C) primary cultured retinal pigment epithelial cells.

FIG. 13. Bar chart demonstrating the percentage resurfacing of AMD afflicted Buch's membrane by retinal pigment epithelial cells with or without treatment with indole-3-carbinol.

FIGS. 14A and 14B. Western blot of integrin α5, integrin β5, integrin β1, integrin β3 and GAPDH protein levels in the macular region of normal (control) donor eye and donor eyes afflicted with age-related macular degeneration (14A) and in the peripheral region of normal (control) donor eye and donor eyes afflicted with age-related macular degeneration (14B).

FIG. 15. Bar chart demonstrating YAP expression levels as fold change from HPRT expression. Q3G and Quercetin-3G denote quercetin-3-glucoside. KFR denotes kaempferol 3-O-β-rutinoside.

FIG. 16. Bar chart demonstrating TAZ expression levels as fold change from HPRT expression. Q3G and Quercetin-3G denote quercetin-3-glucoside. KFR denotes kaempferol 3-O-β-rutinoside.

FIG. 17. Bar chart demonstrating IFN-β1 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 18. Bar chart demonstrating IL-6 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 19. Bar chart demonstrates CXCL-1 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 20. Bar chart demonstrates CXCL-10 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 21. Bar chart demonstrating IFN-β1 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 22. Bar chart demonstrates IL-6 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 23. Bar chart demonstrates CXCL-1 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIG. 24. Bar chart demonstrates CXCL-10 expression levels as fold change from GAPDH expression. Q3G denote quercetin-3-glucoside. K3R denotes kaempferol 3-O-β-rutinoside. GA denotes gallic acid. I3C denotes indole-3-carbinol.

FIGS. 25A, 25B and 25C. Cell adhesion on 0.2 kPa Cytosoft® plates as untreated (25A), AREDS (25B) and ATORC (25C).

### DETAILED DESCRIPTION OF THE INVENTION

The Applicant has surprisingly discovered that one or more of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof treat dry eye disease and/or age-related macular degeneration, increase Bruch's membrane stiffness, and adhesion of retinal pigment epithelial cells to a Bruch's membrane and modulate the expression of YAP, TAZ, integrin α5, integrin β5, interferon β1, interleukin-6, C-X-C motif chemokine ligand 1, and/or C-X-C motif chemokine ligand 10.

Not to be held to a particular theory, the Applicant hypothesizes that disruption of adhesion of retinal pigment epithelial (“RPE”) cells to the Bruch's membrane is a major cause of age-related macular degeneration (“AMD”). The Applicant further theorizes that many mechanosensors that target downstream genes YAP and TAZ including integrins, especially integrins α5 and β5 mediate the adhesion of the RPE cell layer to the Bruch's membrane. The Applicant further theorizes that changes in the stiffness of the Bruch's membrane may also affect the adhesion of RPE cells to the Bruch's membrane leading to AMD.

The Applicant has discovered that expression of integrins α5 and β5 are downregulated in the macular region RPE cells and are not downregulated in peripheral region RPE cells. The Applicant has also discovered that YAP and TAZ expression is decreased in geography atrophy (“GA”). GA is a chronic progressive degeneration of the macula in late-stage AMD. Finally, the Applicant has discovered that the stiffness of the Bruch's membrane significantly decreases as compared to a normal eye in early AMD and significantly increases as compared to a normal eye in GA.

The Applicant has also further and surprisingly discovered that that one or more of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof modulates the secretory profile of senescent cells exhibiting the senescence-associated secretory phenotype (“SASP”) prior to administration of the one or more active ingredients. The secretory profile of SASP includes expression and/or secretion of factors such as interferon β1, interleukin-6, C-X-C motif chemokine ligand 1, and/or C-X-C motif chemokine ligand 10.

It is a further theory of the present invention that maintaining mechanotransduction (i.e. the conversion of mechanical stimuli into electrochemical activity) through YAP and TAZ suppresses aberrant activation of GMP-AMP (“cGAS”) and Stimulator of Interferon Genes (“STING”) signaling. cGAS-STING signaling has been the leading inducer of SASP factors including inflammatory cytokines such as interleukins and type 1 interferons, through response to cues from the cell's microenvironment. Thus, maintenance of YAP/TAZ mechanotransduction ultimately leads to modulation of SASP.

Further, SASP is often exacerbated during decreased adhesion of RPE cells to the Bruch's membrane. Not to be held to a particular theory, SASP contributes significantly to the progression of AMD by promoting inflammation, tissue degradation, and further cellular detachment. The methods of the present invention not only improve cellular adhesion but also mitigate the adverse effects of SASP through modulation of gene expression including but not limited to YAP, TAZ, integrin α5, integrin β5 interferon β1, interleukin-6, C-X-C motif chemokine ligand 1, and/or C-X-C motif chemokine ligand 10. This dual-action approach of increasing cell adhesion and modulating SASP prevents, treats and potentially reverses the pathophysiological changes associated with AMD.

In one embodiment, the present invention is directed to a composition comprising two or more active ingredients selected from the group consisting of quercetin, metabolites, derivatives and salts and hydrates thereof, a kaempferol rutinoside and salts and hydrates thereof, indole-3-carbinol and derivatives and metabolites and salts and hydrates thereof and gallic acid and salts and hydrates thereof.

In a preferred embodiment, the compositions of the present invention are prepared for oral administration.

In another embodiment, the present invention is directed to methods for treating dry eye disease comprising administering a composition of the present invention to a subject in need thereof.

In another embodiment, the present invention is directed to methods for treating dry eye disease comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

In another embodiment, the present invention is directed to methods for treating age-related macular degeneration comprising administering a composition of the present invention to a subject in need thereof.

In another embodiment, the present invention is directed to methods for treating age-related macular degeneration comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

In a preferred embodiment, the age-related macular degeneration being treated is dry age-related macular degeneration.

In another preferred embodiment, the age-related macular degeneration being treated in wet age-related macular degeneration.

In another embodiment, the present invention is directed to methods for increasing Bruch's membrane stiffness comprising administering a composition of the present invention to a subject in need thereof.

In another embodiment, the present invention is directed to methods for increasing Bruch's membrane stiffness comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

In a preferred embodiment, the Bruch's membrane stiffness is increased during early age-related macular degeneration. As used herein, the term “early age-related macular degeneration” refers to an initial stage of age-related macular degeneration characterized by the presence of drusen, which are yellow deposits under the retina measuring less than 125 microns in diameter.

In another embodiment, the present invention is directed to methods for modulating expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof comprising administering a composition of the present invention to a subject in need thereof.

In another embodiment, the present invention is directed to methods for modulating expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

In a preferred embodiment, the subject in need of modulation in expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof is suffering from age-related macular degeneration.

In another embodiment, the present invention is directed to methods for increasing adhesion of retinal pigment epithelium cells to a Bruch's membrane comprising administering a composition of the present invention to a subject in need thereof.

In another embodiment, the present invention is directed to methods for increasing adhesion of retinal pigment epithelium cells to a Bruch's membrane comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

In a preferred embodiment, the subject in need of an increase in adhesion of retinal pigment epithelium cells to a Bruch's membrane is suffering from age-related macular degeneration.

In another embodiment, the present invention is directed to methods of modulating a secretory profile of senescent cells in a subject comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof, wherein the senescent cells of the subject display the senescence-associated secretory phenotype prior to administration of the one or more active ingredients.

Administration of mixtures of the present invention may occur orally.

Kaempferol (CAS #520-18-3), also known as 3,5,7-trihydroxy-2-(4-hydroxyphenyl) chromen-4-one has the following chemical structure

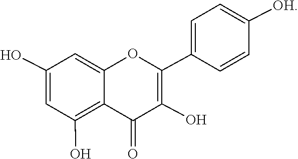 [SMILES: O=c1c(O)c(-c2ccc(O)cc2)oc2cc(O)cc(O)c12]

Kaempferol derivatives include, but are not limited to, kaempferol 3-O-[(6-O-E-caffeoyl)-β-D-glucopyranosyl-(1→2)]-β-D-galactopyranoside-7-O-β-D-glucuropyranoside, kaempferol 3-O-[(6-O-E-p-coumaroyl)-β-D-glucopyranosyl-(1→2)]-β-D-galactopyranoside-7-O-β-D-glucuropyranoside, kaempferol 3-O-[(6-O-E-feruloyl)-β-D-glucopyranosyl-(1→2)]-β-D-galactopyranoside-7-O-β-D-glucuropyranoside, kaempferol 3-O-β-d-(6′-galloyl) glucopyranoside, Kaempferol 5-O-β-D-glucopyranoside, kaempferol 7-O-α-L-rhamnopyranoside, kaempferol 3-O-α-L-arabinopyranoside-7-O-α-L-rhamnopyranoside, kaempferol 3-O-β-rutinoside, kaempferol 3-neohesperidoside, kaempferol 3-O-[rhamnosyl-glucosylglucoside] 7-O-rhamnoside, kaempferol-4′,7-dirhamnoside, Kaempferol-3-O-(2,6-di-p-coumaroyl)-glucoside, Kaempferol-3-O-(3,4-diacetyl-2,6-di-p-coumaroyl)-glucoside, kaempferol 3-O-methyl ether, kaempferol 3-O-α-(4″-O-acetyl) rhamnopyranoside, kaempferol 3-O-α-(3″-O-acetyl) rhamnopyranoside, kaempferol 3-O-α-(3″,4″-di-O-acetyl) rhamnopyranoside, kaempferol 3-O-α-L-(2-acetyl) rhamnopyranoside-7-O-α-L-rhamnopyranoside, kaempferol 3-O-α-L-(3-acetyl) rhamnopyranoside-7-O-α-L-rhamnopyranoside, kaempferol 3-O-α-L-(4-acetyl) rhamnopyranoside-7-O-α-L-rhamnopyranoside, kaempferol 3-O-α-D-glucopyranoside-7-O-α-L-rhamnopyranoside, kaempferol 3-O-[(6-O-E-feruloyl)-β-d-glucopyranosyl-(1→2)]-β-d-galactopyranoside-7-O-β-d-glucuropyranoside, kaempferol 3-O-{[(6-O-E-p-coumaroyl)-β-d-glucopyranosyl (1→2)]-α-1-rhamnopyranosyl-(1→6)}-β-d-galactopyranoside-7-O-α-1-rhamnopyranoside) and kaempferol 3-O-[(6-O-E-caffeoyl)-β-d-glucopyranosyl-(1→2)]-β-d-galactopyranoside-7-O-(2-O-E-caffeoyl′)-β-d-glucuropyranoside. In a preferred embodiment, the kaempferol derivative is kaempferol 3-O-β-rutinoside

Kaempferol 3-O-β-rutinoside (CAS #17650-84-9), also known as nicotiflorin, has the following chemical structure

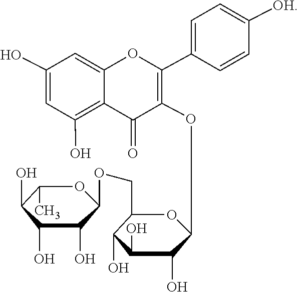

Quercetin (CAS #117-39-5) has the following chemical structure

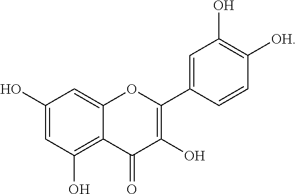 [SMILES: O=c1c(O)c(-c2ccc(O)c(O)c2)oc2cc(O)cc(O)c12]

Quercetin 3-beta-O-glucoside (CAS #482-35-9) has the following chemical structure

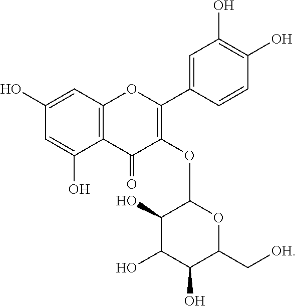 [SMILES: O=c1c(OC2OC(CO)[C@@H](O)C(O)[C@H]2O)c(-c2ccc(O)c(O)c2)oc2cc(O)cc(O)c12]

Indole-3-carbinol (CAS #458-37-7) has the following chemical structure

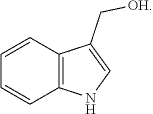 [SMILES: OCc1c[nH]c2ccccc12]

Derivatives and metabolites of indole-3-carbinol include, but are not limited to, indole [3,2-b]carbazole (ICZ), indole-3-acetonitrile, and 3,3′-diindolylmethane (DIM).

Gallic acid (CAS #149-91-7) has the following chemical structure

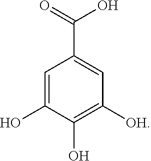 [SMILES: O=C(O)c1cc(O)c(O)c(O)c1]

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

In a preferred embodiment, the compositions of the present invention are oral compositions.

Oral compositions of the present invention may further comprise oils, fatty acids, vitamins and minerals.

Oils suitable for use in oral compositions of the present invention include, but are not limited to, fish oil, flaxseed oil and the like.

Fatty acids suitable for use in oral compositions of the present invention include, but are not limited to, omega-3 fatty acids such as linolenic acid, eicosapentaenoic acid, docosahexaenoic acid and the like.

Vitamins suitable for use in oral compositions of the present invention include, but are not limited to, vitamin A, beta carotene, vitamin B2, vitamin B12, vitamin C, vitamin E and the like.

Minerals suitable for use in oral compositions of the present invention include, but are not limited to, zinc, selenium, copper, manganese and the like and salts thereof.

Oral compositions of the present invention may be mixed into a single composition with AREDS. Further, oral compositions of the present invention may be administered concurrently or sequentially with AREDS.

As used herein the term “AREDS” refers to a composition comprising vitamin C, vitamin E, copper (as cupric oxide), zinc, lutein and zeaxanthin.

In another preferred embodiment, the compositions of the present invention may further comprise lutein and/or zeaxanthin.

In another preferred embodiment, the methods of the present invention may further comprise the co-administration of lutein and/or zeaxanthin including concurrent and sequential administration.

In another preferred embodiment, the compositions of the present invention are ophthalmic compositions.

Ophthalmic compositions of the present invention may further comprise carriers, surfactants, viscosity enhancers, tonicity adjustors, preservatives, antioxidants and buffers.

As used herein the term “modulating expression” refers to increasing, inducing, promoting, and/or activating expression of a gene. The term “modulating expression” also refers to reducing, silencing, and/or inhibiting expression of a gene. The modulation of gene expression may occur via direct interaction with the gene and/or its regulatory elements including cis- and trans-regulatory elements. The modulation of gene expression may be achieved by any pharmacological means available including but not limited to transcriptional gene modulation such as triplex-forming oligonucleotides, synthetic polyamides, designer zin-finger proteins and the like; post-transcriptional gene modulation such as RNAi and the like.

As used herein the term “YAP” refers to a yes-associated protein, the gene coding for a yes-associated protein or a messenger RNA transcribed from a gene coding for a yes-associated protein.

As used herein the term “TAZ”, refers to a transcriptional coactivator with PDZ-binding motif, the gene coding for a transcriptional coactivator with PDZ-binding motif or a messenger RNA transcribed from a gene coding for a transcriptional coactivator with PDZ-binding motif.

As used herein the term “integrin α5”, refers to the integrin alpha 5 protein, the gene coding for the integrin alpha 5 protein or a messenger RNA transcribed from a gene coding for the integrin alpha 5 protein.

As used herein the term “integrin β5”, refers to the integrin beta 5 protein, the gene coding for the integrin beta 5 protein or a messenger RNA transcribed from a gene coding for the integrin beta 5 protein.

As used herein the term “interferon β1” or “IFN-β1”, refers to the interferon β1 protein, the gene coding for the interferon β1 protein or a messenger RNA transcribed from a gene coding for the interferon β1 protein.

As used herein the term “interleukin-6” or “IL-6”, refers to the interleukin-6 protein, the gene coding for the interleukin-6 protein or a messenger RNA transcribed from a gene coding for the interleukin-6 protein.

As used herein the term “C-X-C motif chemokine ligand 1” or “CXCL-1”, refers to the C-X-C motif chemokine ligand 1 protein, the gene coding for the C-X-C motif chemokine ligand 1 protein or a messenger RNA transcribed from a gene coding for the C-X-C motif chemokine ligand 1 protein.

As used herein the term “C-X-C motif chemokine ligand 10” or “CXCL-10”, refers to the C-X-C motif chemokine ligand 10 protein, the gene coding for the C-X-C motif chemokine ligand 10 protein or a messenger RNA transcribed from a gene coding for the C-X-C motif chemokine ligand 10 protein.

As used herein the term “treatment” or “treating” refers to preventing or slowing the progression of or reversing the progression of dry eye disease and/or age-related macular degeneration or reducing, alleviating or ameliorating the symptoms of dry eye disease and/or age-related macular degeneration. In a preferred embodiment, the term “treatment” or “treating” refers to slowing the progression of dry eye disease and/or age-related macular degeneration.

As used herein the term “effective amount” refers to the amount necessary to treat a patient in need thereof.

Throughout the application, the singular forms “a,” “an,” and “the” include plural reference unless the context clearly dictates otherwise.

Throughout the application, all disclosed ranges include all possible values within those ranges. All possible values within the ranges disclosed in the application can also be used as endpoints for additional ranges between these values.

The disclosed embodiments are simply exemplary embodiments of the inventive concepts disclosed herein and should not be considered as limiting unless the claims expressly state otherwise.

The following example is intended to illustrate the present invention and to teach one of ordinary skill in the art how to use the methods of the invention. The example is not intended to be limiting in any way.

### EXAMPLES

### Example 1—Treatment of Dry Eye Disease

### Method

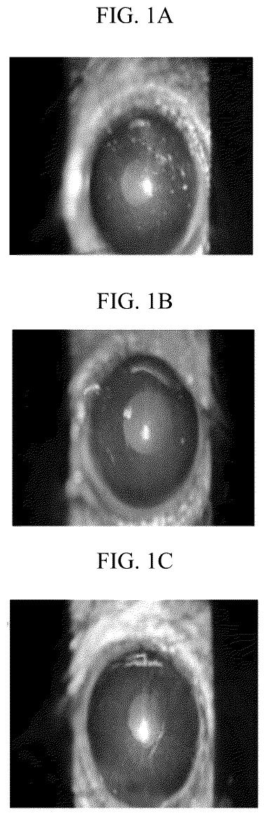

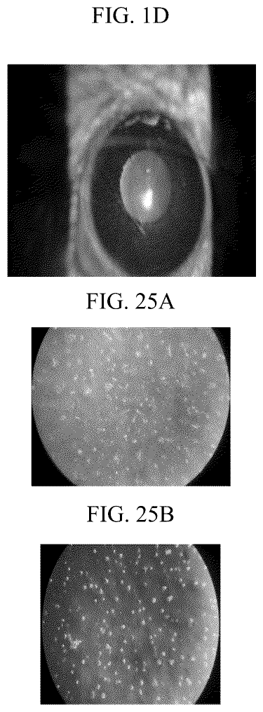

Dry eye disease was induced in mice by administering subcutaneous injections of 0.1 milliliters of scopolamine at 5 milligrams per milliliter three times per day for 14 days. Concurrently, indole-3-carbinol was instilled in the right eye of the mice at either 100 micromolar or 1 millimolar. As a control saline was instilled in the left eye of the mice. Following instillation, a baseline Schirmer's test (tear volume) was performed. On the 14th day, corneal epithelial disruption was assessed using slit lamp analysis following 1% fluorescein staining. Subsequently, the mice were euthanized via ketamine overdose, and their eyes were enucleated, with the corneas isolated under sterile conditions. See FIG. 1 for results.

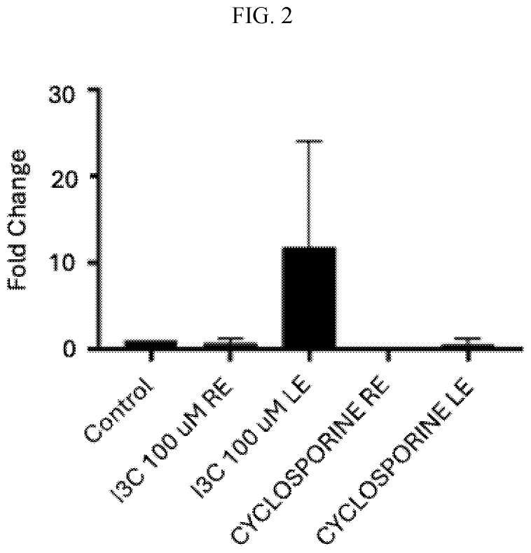

Intercellular adhesion molecule 1 (“ICAM-1”) plays a pivotal role in instigating ocular surface inflammation during hyperosmolar stress. The involvement of ICAM-1 in exacerbating the inflammatory cascade, leading to the subsequent release of cytokines, underscores its significance in dry eye disease. Thus, elevated ICAM-1 levels indicates induction of dry eye disease. Whereas reduction of ICAM-1 levels from the induced state demonstrates treatment of dry eye disease. Further, inhibition of ICAM-1 activation, prevents immune cell accumulation and adhesion on ocular surface, thereby reducing inflammation. Accordingly, mRNA expression for ICAM-1, pro-inflammatory marker, was also assessed for the mice treated with 100 micromolar of indole-3-carbinol. As a positive control a set of mice were also treated with cyclosporine. See FIG. 2 and Table 1, below, for results.

TABLE 1

| Treatment | Fold Change from Control |
|---|---|
| Control | 1.00 |
| 100 μM indole-3-carbinol right eye | 0.74 |
| 100 μM indole-3-carbinol left eye | 11.78 |
| Cyclosporine right eye | 0.19 |
| Cyclosporine left eye | 0.62 |

### Results

As demonstrated in FIG. 1, mouse eyes treated with either 100 micromolar or 1 millimolar of indole-3-carbinol demonstrated less disruption of the corneal epithelium than mouse eyes treated with saline. Compare 1b with 1a and 1d with 1c.

As depicted in FIG. 2, levels of ICAM-1 are elevated in eyes induced with the disease, but are relatively reduced in eyes treated with indole-3-carbinol at 100 micromolar. Thus, indole-3-carbinol is effective at treating dry eye disease.

### Example 2—Bruch's Membrane Stiffness in Age-Related Macular Degeneration

### Method

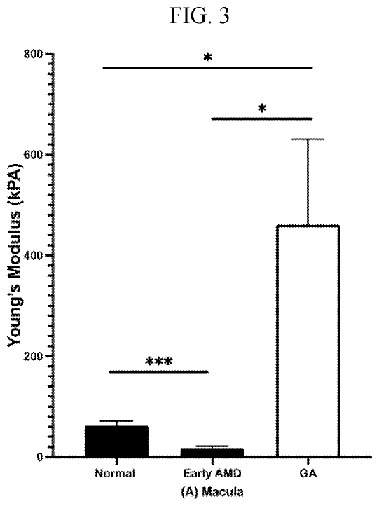

Bruch's membrane stiffness was measured using atomic force microscopy in the macular region of a normal eye, an eye under early AMD and an eye under geographic atrophy (“GA”). Results of this study can be found in FIG. 3.

### Results

As demonstrated in FIG. 3, the stiffness of the Bruch's membrane in the macular region of a normal eye is about 60 kiloPascals (“kPA”), in an eye under early AMD is about 20 kPA and in an eye under GA is about 460 kPA. Not to be held to a particular theory, the reduced stiffness of the Bruch's membrane during early AMD contributes to the decrease in adhesion of retinal pigment epithelial cells to the Bruch's membrane.

### Example 3—RPE Cell Adhesion in Age-Related Macular Degeneration

### Method

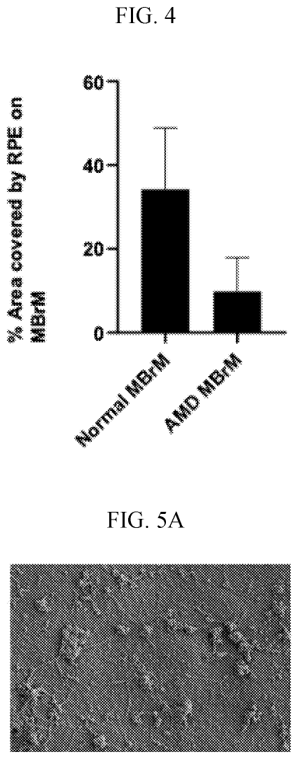

Retinal pigment epithelial (“RPE”) cell adhesion to the macular region of the Bruch's membrane was measured as a percent area coverage of RPE cells on the membrane in a normal eye and an eye under AMD. Results of this study can be found in FIG. 4.

### Results

As demonstrated in FIG. 4, the percent area of the macular region of the Bruch's membrane covered by RPE cells in a normal eye was about 35% and in an eye under AMD was about 10%.

### Example 4—Treatment of Age-Related Macular Degeneration

### Method

Bruch's membranes extracted from human cadaver eyes was seeded with retinal pigment epithelial (“RPE”) cells that were either untreated or treated with kaempferol 3-O-β-rutinoside (“KFR”) and allowed to re-attach for seven days. One of the chief causes for age-related macular degeneration progression is the loss of RPE cells from Bruch's membrane due to several underlying factors including structural changes of RPE cells, thickening of Bruch's membrane, drusen formation etc. Thus, an increase in RPE cell attachment indicates an effective treatment for age-related macular degeneration.

### Results

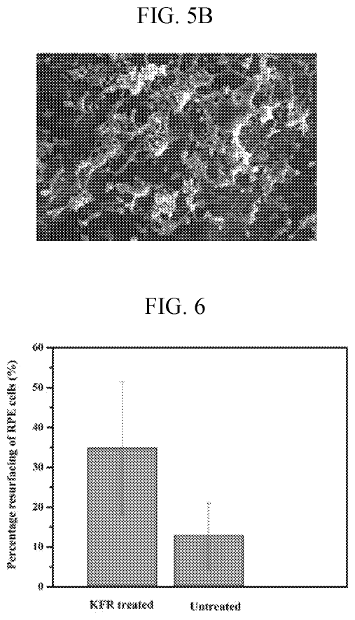

Our preliminary studies have shown that there is significant increase in RPE cell resurfacing of Buch's membranes when treated with KFR, compared to control. See the 5000× magnification Scanning Electron Microscope (“SEM”) images of the Bruch's membranes in FIG. 5b, treated as compared to FIG. 5a, control. Specifically, 35% of the surface of Buch's membrane was resurfaced with RPE cells following treatment with KFR compared to only 13% for control. See FIG. 6. Thus, it is evident that KFR can be effectively used in controlling age-related macular degeneration.

### Example 5—Treatment of Age-Related Macular Degeneration

### Method

Both normal and AMD afflicted Bruch's membranes were extracted from human cadaver eyes was seeded with RPE cells that were either untreated or treated with a mixture of KFR, quercetin-3-glucoside and gallic acid (“the mixture”) at 1 micromolar (“μM”) or 10 nanomolar (“nM”) and allowed to re-attach for six days.

### Results

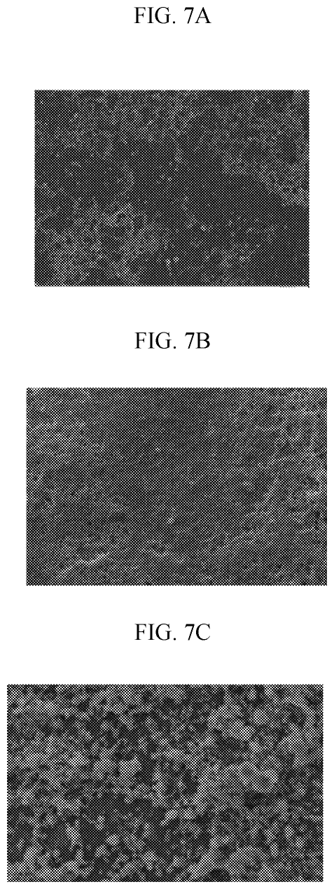

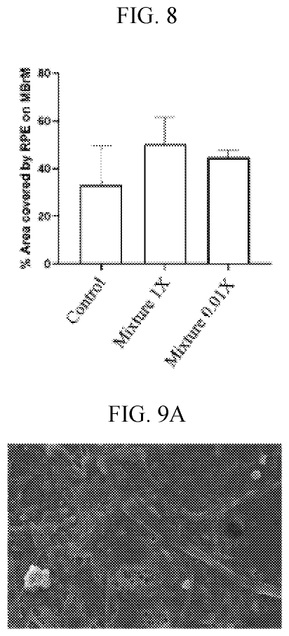

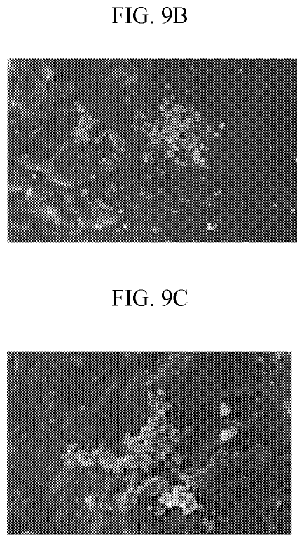

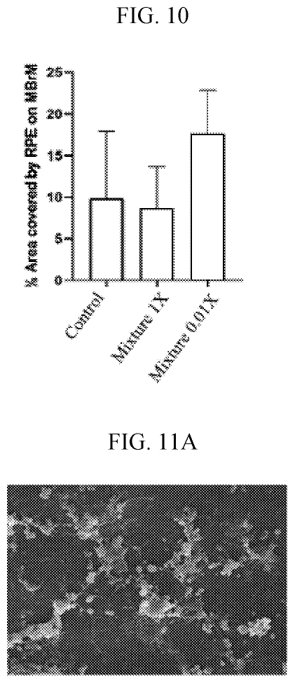

Our preliminary studies have shown that there is significant increase in RPE cell resurfacing of both normal and AMD afflicted Bruch's membranes when treated with the mixture at either 1 μM or 10 nM compared to control. See the 5000× magnification SEM images of the normal Bruch's membranes in FIGS. 7b and 7c, respectively, treated as compared to FIG. 7a, control. Specifically, 50% of the surface of normal Buch's membrane was resurfaced with RPE cells following treatment with the mixture at 1 μM and 44% when treated with the mixture at 10 nM compared to only 36% for control. See FIG. 8. See further, the 5000× magnification SEM images of the AMD afflicted Bruch's membranes in FIGS. 9b and 9c, treated as compared to FIG. 9a, control. Specifically, 8.7% of the surface of AMD afflicted Bruch's membrane was resurfaced with RPE cells following treatment with the mixture at 1 μM and 17% when treated with the mixture at 10 nM compared to only 10% for control. See FIG. 10.

Thus, it is evident that a mixture of kaempferol 3-O-β-rutinoside, quercetin-3-glucoside and gallic acid can be effectively used in controlling age-related macular degeneration.

### Example 6—Treatment of Age-Related Macular Degeneration

### Method

Both normal and AMD afflicted Bruch's membranes were extracted from human cadaver eyes was seeded with RPE cells that were either untreated or treated with indole-3-carbinol 1 μM or 10 nM and allowed to re-attach for six days.

### Results

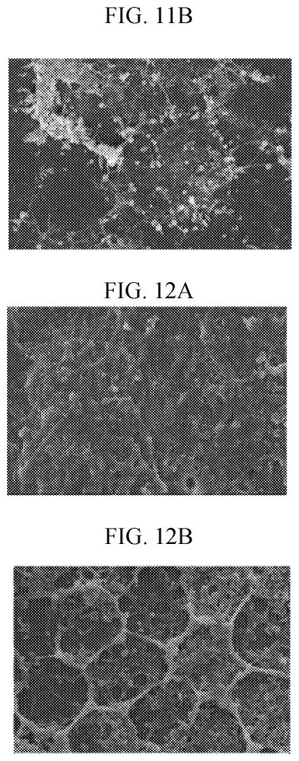

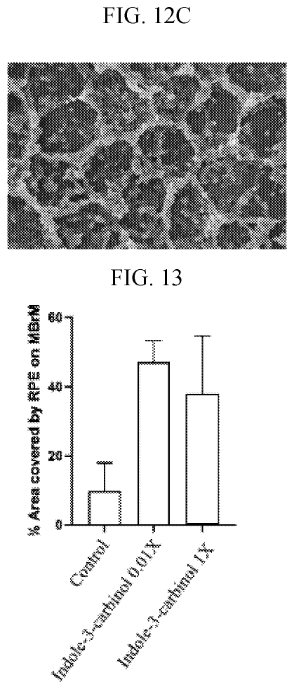

Our preliminary studies have shown that there is significant increase in RPE cell resurfacing of AMD afflicted Bruch's membranes when treated with indole-3-carbinol at either 1 μM or 10 nM compared to control. See the 5000× magnification SEM images of the normal Bruch's membranes in FIG. 11b, treated as compared to FIG. 11a, control. See also, the 5000× magnification SEM images of the AMD afflicted Bruch's membranes demonstrates an increase in RPE cell resurfacing when treated with indole-3-carbinol as seen in FIGS. 12b and 12c, treated, as compared to FIG. 12a, control. Specifically, 38% of the surface of AMD afflicted Bruch's membrane was resurfaced with RPE cells following treatment with indole-3-carbinol at 1 μM and 47% when treated with indole-3-carbinol at 10 nM compared to only 10% for control. See FIG. 13.

Thus, it is evident that indole-3-carbinol can be effectively used in controlling age-related macular degeneration.

### Example 7—Downregulation of Integrin α5 and Integrin β5 in Macular Region of AMD-Afflicted Eyes

### Method

Macular and peripheral retinal pigment epithelium tissues were collected from normal and donor eye and donor eyes afflicted with age-related macular degeneration. Total protein was isolated and quantified using a standard bicinchoninic acid assay (BCA) kit. Immunoblotting was performed for both integrin α5 and integrin β5, with glyceraldehyde 3-phosphate dehydrogenase (GAPDH) as the internal control.

### Results

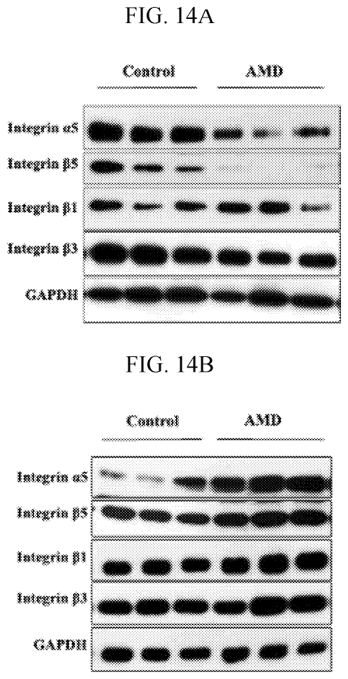

As demonstrated in FIG. 14a, both integrins were downregulated in macular RPE tissues under AMD conditions. In contrast, the peripheral samples exhibited an opposite expression pattern. See FIG. 14b.

### Example 8—Increased YAP and TAZ Expression

### Method

Yes-associated protein (“YAP”) and a transcriptional co-activator having a PDZ-binding motif (“TAZ”) are transcription co-activators in the Hippo pathway and help regulate cell proliferation, differentiation and migration. Hu, Y. et al., YAP suppresses gluconeogenic gene expression via PGC1alpha, Hepatology, 2017 and Plouffe, S. W. et al., Disease implications of the Hippo/YAP pathway, Trends Mol Med, 2015 (21) 212-222. YAP/TAZ are crucial mechanotransducers which respond to changes in stiffness of the Bruch's membrane and their depletion can induce cellular senescence and release of SASP factors.

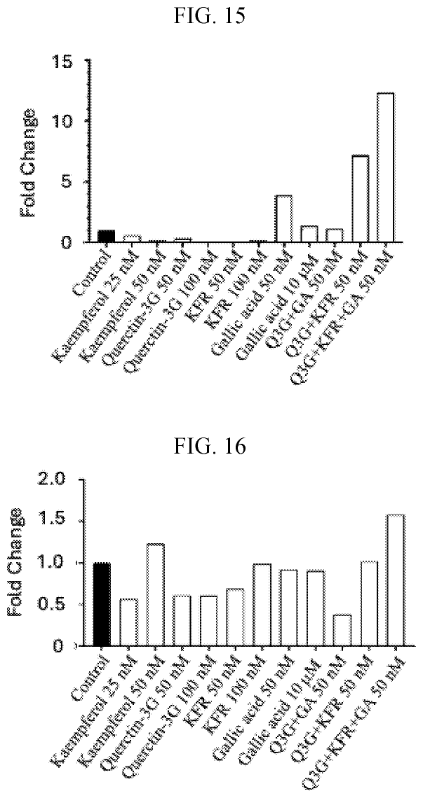

Human RPE cells were seeded onto a substrate having a stiffness of 2 kPA, which reproduces the stiffness of AMD afflicted Bruch's membranes during early AMD. These cells were then treated with either 1) control vehicle, 2) 25 nanomolar (“nM”) kaempferol, 3) 50 nM kaempferol, 4) 50 nM quercetin-3-glucoside, 5) 100 nM quercetin-3-glucoside, 6) 50 nM KFR, 7) 100 nM KFR, 8) 50 nM gallic acid, 9) 10 micromolar (“μM”) gallic acid, 10) a mixture of quercetin-3-glucoside and gallic acid at 50 nM, 11) a mixture of quercetin-3-glucoside and KFR at 50 nM or 12) a mixture of quercetin-3-glucoside, KFR and gallic acid at 50 nM. YAP and TAZ expression levels were determined via RT-PCR and are presented in FIGS. 15 and 16, respectively and also in Table 2, below. Expression levels are presented as fold change from HPRT expression.

Synergy was calculated by dividing the observed fold change in expression of either YAP or TAZ following application of each mixture by the expected fold change in expression, wherein a synergy factor of greater than 1 denotes synergy. Expected fold change in expression was calculated using the following formula (A+B)−(A*B), wherein A and B denote the observed fold change in expression following treatment with either of the actives alone.

TABLE 2

| | | | Synergy | | Synergy |
|---|---|---|---|---|---|
| Treatment | Concentration | YAP | Factor | TAZ | Factor |
| Control | — | 1 | — | 1 | — |
| Kaempferol | 25 nM | 0.59 | — | 0.56 | — |
| Kaempferol | 50 nM | 0.23 | — | 1.23 | — |
| Quercetin-3-glucoside | 50 nM | 0.35 | — | 0.61 | — |
| Quercetin-3-glucoside | 100 nM | 0.07 | — | 0.61 | — |
| KFR | 50 nM | 0.03 | — | 0.69 | — |
| KFR | 100 nM | 0.23 | — | 0.99 | — |
| Gallic acid | 50 nM | 1.36 | — | 0.91 | — |
| Gallic acid | 10 μM | 3.86 | — | 0.92 | — |
| Quercetin-3-glucoside | 50 nM | 1.13 | 0.91 | 0.38 | 0.4 |
| Gallic acid | 50 nM | | | | |
| Quercetin-3-glucoside | 50 nM | 7.16 | 25.46 | 1.02 | 1.02 |
| KFR | 50 nM | | | | |
| Quercetin-3-glucoside | 50 nM | 12.32 | 7.13 | 1.58 | 0.87 |
| KFR | 50 nM | | | | |
| Gallic acid | 50 nM | | | | |

### Results

As demonstrated in Table 2 and FIGS. 15 (YAP) and 16 (TAZ), binary and ternary mixtures of quercetin-3-glucoside, KFR and gallic acid increase YAP and TAZ expression in human RPE cells affixed to a stiff substrate. These results indicate that binary and ternary mixtures of the present invention can treat AMD. Further, these results indicate that these mixtures can produce synergistic treatment of AMD.

### Example 9—Decreased SASP Factor Expression

Interferon β1 (“IFN-β1”) and interleukin-6 (“IL-6”) are cytokines secreted during cellular senescence (i.e. ceasing of cell division) and activate cGAS-STING pathway, which augments the inflammatory response. C-X-C motif chemokine ligand 1 (“CXCL-1”) and C-X-C motif chemokine ligand 10 (“CXCL-10”) are chemokines that are also part of the SASP and influence immune cell infiltration and inflammation. Mechanosensing (i.e. the sensing of mechanical cues from the cells microenvironment) links mechanotransducers such as YAP/TAZ to SASP factors such as IFN-β1, IL-6, CXCL-1, and CXCL-10. The subsequent collective inhibition of these SASP factors through modulation of YAP/TAZ expression contributes to increased RPE cell adhesion to Bruch's membrane.

### Method

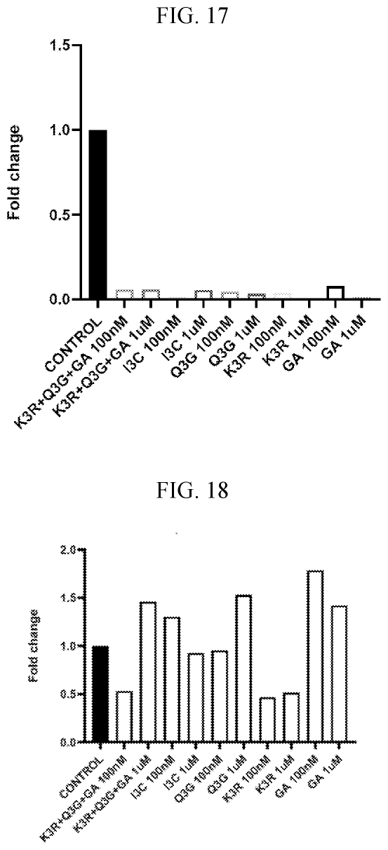

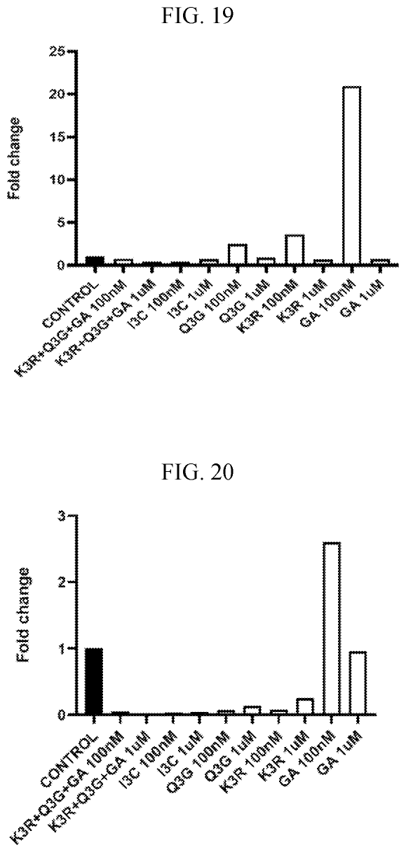

Human RPE cells were seeded onto a substrate having a stiffness of 0.2 kPA, which reproduces the stiffness of AMD afflicted Bruch's membranes during early AMD. These cells were then treated with either 1) control vehicle, 2) a mixture of quercetin-3-glucoside, kaempferol 3-O-β-rutinoside and gallic acid at 100 nM, 3) a mixture of quercetin-3-glucoside, kaempferol 3-O-β-rutinoside and gallic acid at 1 μM, 4) indole-3-carbinol at 100 nM, 5) indole-3-carbinol at 1 μM 6) quercetin-3-glucoside at 100 nM, 7) quercetin-3-glucoside 1 μM, 8) kaempferol 3-O-β-rutinoside at 100 nM, 9) kaempferol 3-O-β-rutinoside at 1 μM, 10) gallic acid at 100 nM, or 11) gallic acid at 1 μM. IFN-β1, IL-6, CXCL-1, and CXCL-10 expression levels with glyceraldehyde 3-phosphate dehydrogenase (GAPDH) as the internal control were determined via RT-PCR and are presented in FIGS. 17, 18, 19, and 20, respectively.

### Results

As demonstrated in FIGS. 17, 18, 19, and 20, SASP factors were downregulated by ternary mixtures of quercetin-3-glucoside, kaempferol 3-O-β-rutinoside and gallic acid. These results indicate that ternary mixtures of the present invention can treat AMD.

### Example 10—Decreased SASP Factor Expression

### Method

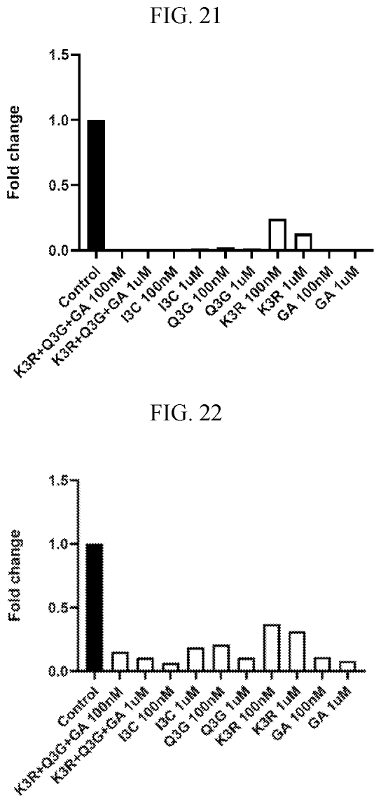

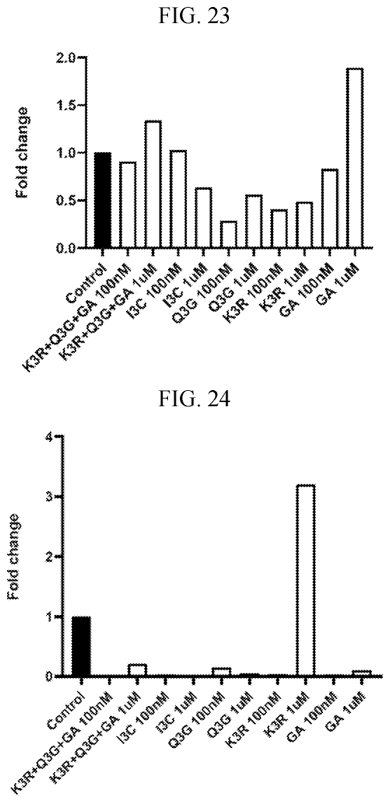

Human RPE cells were seeded onto a substrate having a stiffness of 16 kPA, which reproduces the stiffness of AMD afflicted Bruch's membranes during early AMD. These cells were then treated with either 1) control vehicle, 2) a mixture of quercetin-3-glucoside, kaempferol 3-O-β-rutinoside and gallic acid at 100 nM, 3) a mixture of quercetin-3-glucoside, kaempferol 3-O-β-rutinoside and gallic acid at 1 μM, 4) indole-3-carbinol at 100 nM, 5) indole-3-carbinol at 1 uM, 6) quercetin-3-glucoside at 100 nM, 7) quercetin-3-glucoside 1 μM, 8) kaempferol 3-O-β-rutinoside at 100 nM, 9) kaempferol 3-O-β-rutinoside at 1 μM, 10) gallic acid at 100 nM, or 11) gallic acid at 1 μM. IFN-β1, IL-6, CXCL-1, and CXCL-10 expression levels with glyceraldehyde 3-phosphate dehydrogenase (GAPDH) as the internal control were determined via RT-PCR and are presented in FIGS. 21, 22, 23 and 24, respectively.

### Results

As demonstrated in FIGS. 21, 22, 23 and 24 SASP markers were downregulated by ternary mixtures of quercetin-3-glucoside, kaempferol 3-O-β-rutinoside and gallic acid. These results indicate that ternary mixtures of the present invention can treat AMD.

### Example 11—Bruch's Membrane Cell Adhesion

### Method

In preclinical studies using human retinal pigment epithelial (“hRPE”) cells, hRPE cells were seeded onto a substrate having varying stiffness, including 0.2 kPa, which reproduces the stiffness of AMD afflicted Bruch's membranes during early AMD and 16 kPa. These cells were then treated with the adhesion of hRPE cells across substrates of varying stiffness was evaluated. In this evaluation we treated the cells with a mixture of kaempferol-3-O-rutinoside, quercetin 3-β-D-glucoside, gallic acid, and indole-3-carbinol (“ATORC”), at concentrations of 100 nM and 1 μM. These results were compared to the effects of the AREDS formulation, used at a 1× concentration. Zeng et al. Effects of Antioxidant Components of AREDS Vitamins and Zinc Ions on Endothelial Cell Activation: Implications for Macular Degeneration, Invest Ophthalmol Vis Sci. 2012 Feb. 27; 53 (2): 1041-1047.

### Results

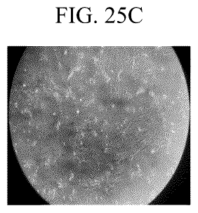

Treatment with ATORC showed increased cell adhesion on 0.2 kPa Cytosoft plates, as observed visually (FIG. 25c). In the control wells, cells appeared clustered and adhered together (FIG. 25a), whereas in the AREDS 1× treated wells, the cells were attached individually (FIG. 25b). Conversely, in the ATORC treated wells, the cells were well-interacted and adhered efficiently (FIG. 25c). This observation indicates that ATORC effectively enhances the adhesive properties of hRPE cells despite the stiffness of the substrate. These findings are consistent with our scanning electron microscopy results of normal and AMD Bruch's membrane.

Additionally, RT-PCR analysis revealed elevated expression of the mechanotransduction regulators YAP and TAZ in cells cultured on both 0.2 kPa and 16 kPa substrates, compared to untreated control cells. Furthermore, the expression of senescence-associated secretory phenotype (“SASP”) markers, including IFN-β1, IL-6, CXCL-1, and CXCL-10, was assessed by RT-PCR. The results indicated a decrease in the expression of these markers in the drug-treated groups compared to the control. These results indicate that ternary mixtures of the present invention can treat AMD.

Not to be held to a particular theory, mixtures of kaempferol, quercetin and gallic acid have free radical scavenging properties. Indole-3-carbinol additionally has the capability to neutralize reactive oxygen species and inhibit lipid peroxidation. Further, a combination of AREDS and ATORC will have both the properties of antioxidant activity as well as the additional activity of increasing cell adhesion to membrane and reducing cell senescence.

What is claimed is:

1. A composition for oral administration comprising two or more active ingredients selected from the group consisting of quercetin, metabolites, derivatives and salts and hydrates thereof, a kaempferol rutinoside and salts and hydrates thereof, indole-3-carbinol and derivatives and metabolites and salts and hydrates thereof and gallic acid and salts and hydrates thereof.

2. The composition of claim 1, wherein the kaempferol rutinoside is kaempferol 3-O-β-rutinoside.

3. The composition of claim 1, further comprising one or more excipients selected from the group consisting of oils, fatty acids, vitamins and minerals.

4. A method of treating dry eye disease comprising administration of an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

5. A method of treating age-related macular degeneration comprising administration of an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

6. The method of claim 5, wherein the age-related macular degeneration is wet age-related macular degeneration.

7. The method of claim 5, wherein the age-related macular degeneration is dry age-related macular degeneration.

8. The method of claim 5, wherein administration occurs orally.

9. A method for increasing Bruch's membrane stiffness comprising administration of an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

10. The method of claim 9, wherein the subject in need thereof is a subject suffering from early age-related macular degeneration.

11. The method of claim 9, wherein administration occurs orally.

12. A method for modulating expression of a gene selected from the group consisting of YAP, TAZ, integrin α5, integrin β5, interferon β1, interleukin-6, C-X-C motif chemokine ligand 1, C-X-C motif chemokine ligand 10, and combinations thereof comprising administration of an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

13. The method of claim 12, wherein the subject in need thereof is a subject suffering from age-related macular degeneration.

14. The method of claim 12, wherein administration occurs orally.

15. A method for increasing adhesion of retinal pigment epithelium cells to a Bruch's membrane comprising administration of an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

16. The method of claim 15, wherein the subject in need thereof is a subject suffering from age-related macular degeneration.

17. The method of claim 15, wherein administration occurs orally.

18. A method for modulating a secretory profile of senescent cells exhibiting the senescence-associated secretory phenotype comprising administering an effective amount of one or more active ingredients selected from the group consisting of kaempferol derivatives and salts thereof and hydrates thereof, quercetin metabolites, derivatives and salts and hydrates thereof, indole-3-carbinol and derivatives and metabolites and salts and hydrates thereof and gallic acid and salts and hydrates thereof to a subject in need thereof.

19. The method of claim 18, wherein administration occurs orally.

20. The composition of claim 1, further comprising lutein and/or zeaxanthin.
